(19) United States
(12) Patent Application Publication
(10) Pub. No.: US 2004/0016037 A1
(43) Pub. Date: Jan. 29, 2004

(54) Disposable eye shield

(75) Inventor: Henry L. Griesbach III, Clarkston, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/200,875

(22) Filed: Jul. 23, 2002

(51) Int. Cl. A61F 9/00

(52) U.S. Cl. 2/12

(57) ABSTRACT

An adjustable eye shield adapted to fit over the eyes and at least partially wrap around the face of a user made from a transparent material. The shield being divided into multiple regions and sections having at least one slit in left and right regions. The shield also includes an adjustable fastener having ends that pass through holes in the shield, traverse the slits in the shield and are secured to the shield. When the adjustable fastener is pulled dedicated sections in the regions are adapted to slide one over the other and conform the shield to a user's face.

### BACKGROUND

This invention relates in general to disposable medical products, and in particular to disposable eye shields designed to prevent liquid from contacting the eyes of a wearer.

The wearing of a protective face mask has become standard procedure in many health care and other related activities. The use of a face mask is important, for example, to lab technicians during the conduction of tests, to nurses in the care of contagious patients, to physicians during surgery and to dentists working in a patient's mouth.

With the rapid increase in the spread of infectious diseases such as AIDS, the use of protective equipment, including masks, has become even more important. The Center for Disease Control in Atlanta, Ga., has found that AIDS can be passed by contact with body fluids. Contact of AIDS contaminated body fluids with another person's source of body fluids, i.e. eyes, nose, mouth, etc., can pass the disease.

It is therefore necessary to prevent a patient's body fluids from contacting the eyes, nose and mouth of an attendant.

The typical previously developed protective mask covers the wearer's face from the bridge of the nose to below the chin, and is generally designed to filter out germs and other particulates. Unfortunately, a mask designed to prevent the passage of germs to the respiratory system has little value in preventing liquids from reaching the eyes, and thus normal face masks provide the wearer with no protection against the patient's body fluids.

### SUMMARY OF THE INVENTION

As such, one aspect of the present invention discloses an adjustable eye shield having a flexible, transparent material which is adapted to fit over the eyes and at least partially wrap around the face of a user. The material is divided into different regions. The regions include at least a left region, a right region, and a central region. At least one slit is provided in both the left and the right regions. The slit extends fully across the region thus subdividing the left region into a first and a second section and subdividing the right region into a first and a second section. At least one hole in each of the first sections of the left and the right regions is provided. An adjustable fastener for fastening the eye shield to the head of the user is provided. The adjustable fastener has a plurality of ends. At least one end of the fastener passes through at least one of the holes in the first section of the right region, traverses the slit in the right region, and is fastened to the second section of the right region. At least one other end of the fastener passes through at least one of the holes in the first section of the left region, traverses the slit in the left region, and is secured to the second section of the left region. This enables the first and second sections of each of the left and right regions to slide one over the other.

In another aspect of the present invention an adjustable eye shield is disclosed having a flattened, flexible, transparent sheet material divided into a plurality of regions. The regions include at least a left region, a right region, and a central region. At least one slit is provided extending fully across the left region and subdividing the region into a first and a second section. One of the sections has a hole therethrough. At least another slit is provided which extends fully across the right region subdividing the region into a first and a second section. One of these sections also has a hole therethrough. An adjustable fastener having a plurality of ends is provided, where a separate end selected from this plurality of ends is dedicated to each of the left and right regions so that the dedicated end is affixed to one of the sections within its dedicated region, traverses the slit in the same region, and passes through the hole in the remaining section opposite the slit in the same region. Manipulation of the adjustable fastener causes the first and second sections of each of the left and right regions to slide one over the other causing the material to gradually bend without creasing.

### BRIEF DESCRIPTION OF THE DRAWINGS

FIG. 1 illustrates one embodiment of an eye shield in accordance with the present invention;

FIG. 2 illustrates another embodiment of an eye shield in accordance with the present invention; and

FIG. 3 illustrates an eye shield in accordance with the present invention in use in conjunction with a face mask.

### DESCRIPTION OF THE INVENTION

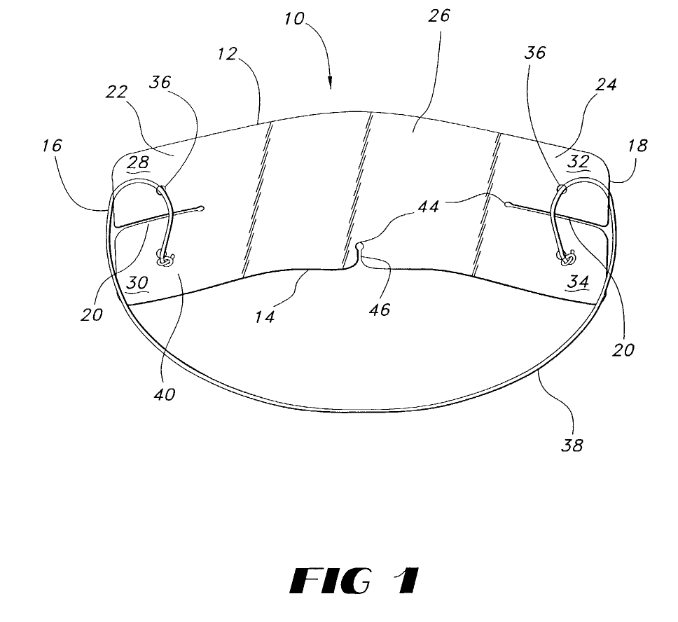

The present invention and its advantages are best understood by referring to FIGS. 1-3 of the drawings, like numerals being used for like and corresponding parts of the various drawings.

Referring first to FIG. 1, an eye shield 10 constructed in accordance with the present invention is shown. The eye shield 10 may comprise a clear flexible, plastic film made from, for instance, polyester or polyethylene. Examples of plastic films suitable for the present invention include oriented polyester films such as Mylar® and Melinex® currently manufactured by Dupont. The film must be stiff enough to prevent collapse yet flexible enough to bend. In one embodiment, the film may have a thickness of approximately 0.1 mm. Other embodiments may range from a thickness of approximately 0.076 mm to approximately 0.18 mm.

In one embodiment, the eye shield 10 may comprise at least a top edge 12, a bottom edge 14, and a left and a right side edge, 16 and 18, respectively. Each of the side edges 16 and 18 contain at least one slit 20 which extends a distance from its respective side edge toward the opposite side edge. This configuration serves to divide the eye shield 10 into at least three regions; a left region 22 and a right region 24 each containing a slit 20, and a central region 26 disposed between the left and right regions. The central region 26, in certain embodiments, is sufficiently wide so as to completely cover and extend a distance beyond the eyes of a user. The left and right regions 22 and 24 respectively are further subdivided lo into first and second sections along the slit 20. That is, the left region 22 comprises a first left section 28 and a second left section 30 separated by slit 20. Similarly the right region 24 comprises a first right section 32 and a second right section 34 also separated by slit 20.

As depicted in FIG. 1, the first sections 28 and 32 may be uppermost, i.e., located proximate to the top edge 12, with respect to second sections 30 and 34 which are located proximate to the bottom edge 14. It should, however, be understood that this configuration is not necessary to the practice of the invention. In fact, the first sections may, in some instances, be more suitably disposed upon the bottom and the second sections would thus be disposed nearer the top. Other possibilities of course exist including subdividing each region 22 and 24 into more than two sections. As such, three or more left sections and three or more right sections may be possible and are thus also contemplated as being described and claimed in the present invention.

Still looking to FIG. 1, it can be seen that the first section 28 and 32 each contain a hole 36 through which an adjustable fastener 38 is passed. The adjustable fastener 38 serves to fasten the eye shield 10 to the head of the user. The fastener 38 may comprise a loop of material such as that shown in FIG. 1 which may or may not be elastic in nature. By the term “elastic”, it is meant that the material is stretchable and includes that property of a material by virtue of which it tends to substantially recover to its original size and shape after removal of a force causing deformation of the material.

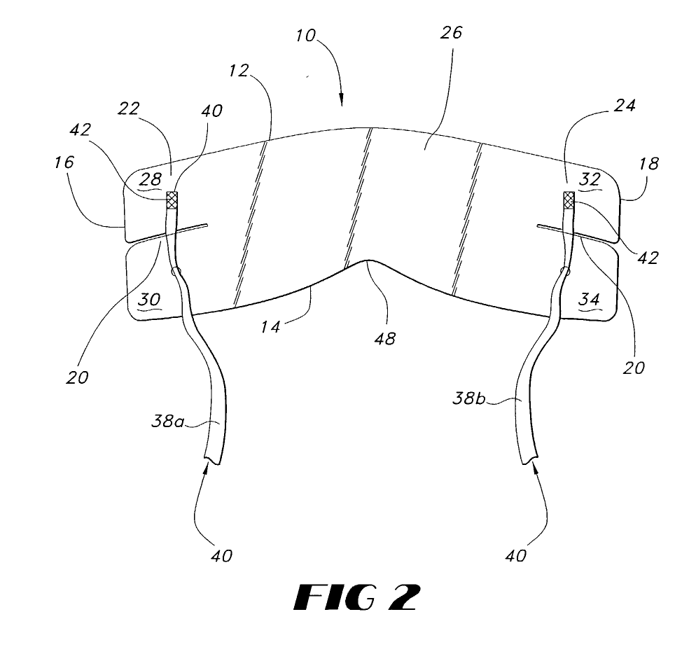

As stated, it can be seen in FIG. 1, that the adjustable fastener 38 comprises a loop of material having a plurality of ends 40, in this case two ends 40. The ends 40 are each affixed to the eye shield 10 at the second sections 30 and 34, respectively. A number of possible fastening methods may be used. For example, as shown in FIG. 1, the ends 40 may be passed through holes similar to holes 36 in sections 28 and 32 and knotted so they do not slip back through the holes. Alternatively, the ends 40 may be fastened to the eye shield by adhesive, heat bond, mechanical fastener, or any other alternative fastening techniques. FIG. 1 also illustrates that one end 40 of the adjustable fastener 38 may be secured to the second section 30 of the left region 22. The fastener 38 then crosses the slit 20 and is threaded through the hole 36 in the first section 28 of the left region 22. The same occurs at the right region 24. The fastener 38 may be positioned on either the front or back of the eye shield 10. Alternatively, the fastener 38 can be passed through the slit 20 from one side of the eye shield 10 to the opposite side as will be described with respect to FIG. 2. Turning now to FIG. 2, the adjustable fastener 38 may comprise a plurality of tie strips such as the two tie strips 38a and 38b depicted. These tie strips may or may not be elastic in nature. It should be understood that the specific number of individual tie strips provided is not limited to the two depicted in FIG. 2. Furthermore, FIG. 2 depicts the ends 40 of the tie strips 38a and 38b bonded to the eye shield 10 at bond points 42.

It should be pointed out that in this embodiment the strips pass from one side of the shield 10 to the opposite through the slits 20. It is also important to note that the FIG. 2 embodiment shows the adjustable fastener 38, in this case tie strips 38a and 38b as being fastened at ends 40 to the first sections 28 and 32 respectively. This configuration serves to illustrate that it is unimportant to the scope of the claims to which sections 28, 30, 32, or 34 that the ends 40 of the adjustable fastener 38 are affixed. It also serves to depict the alternative pathway configurations which the fastener 38 may assume, i.e., to remain completely on one side of the eye shield 10 or to pass through the slit 20 from one side to the other.

FIGS. 1 and 2 also serve to depict differing embodiments regarding the slit 20. In FIG. 1, each slit 20 may comprise a widened portion 44, in this case a circular cutout. This widened portion 44 serves to relieve stresses that may otherwise concentrate at the slit 20 which could lead to premature tearing or deformation of the eye shield 10. However, as shown in FIG. 2, these widened portions 44 are not necessary to the practice of the invention. FIGS. 1 and 2 also illustrate possible alternative embodiments of the bottom edge 14. FIG. 1 depicts a slit 46 adapted to accommodate the user's nose. The slit 46 is configured similar to slits 20 and in FIG. 1 terminates in the optional widened portion 44. FIG. 2 depicts a generally concave cutout 48 also designed to accommodate the user's nose. Other embodiments are of course possible and are readily understood by those skilled in the art.

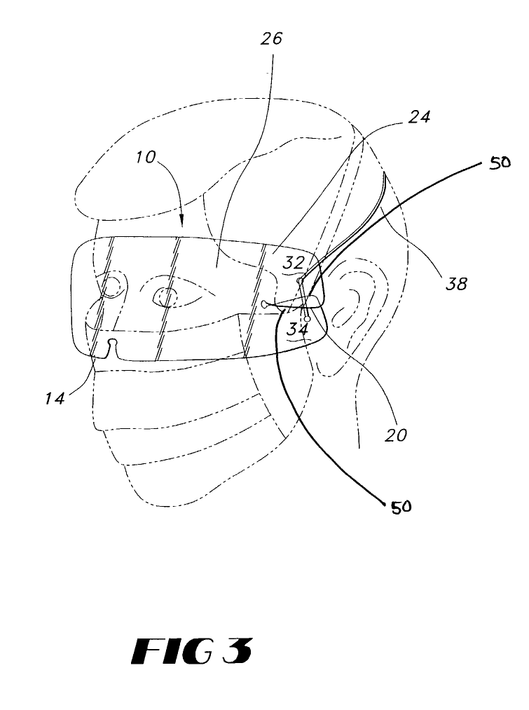

Operation of the device can best be described by turning to FIG. 3. The eye shield 10 itself is adjusted by manipulation of the adjustable fastener 38. First, the eye shield 10 is positioned across a user's face so that the central region 26 covers the eyes and the left and right regions 22 and 24, respectively and partially wraps around to the side of the user's head as shown in the FIG. Next the user pulls on the adjustable fastener 38 or fasteners 38a and 38b, causing the first and second sections 28 and 30 of the left region 22 to slide one over the other. This enables an edge 50 of one section to overlap an edge 50 of the other section allowing an overlapping configuration of the shield 10 at the slit 20. Likewise, the first and second sections 32 and 34 of the right region 24 are caused to slide one over the other in a similar fashion.

As the adjustable fastener 38 is pulled, the end 40 fastened to the eye shield 10 moves closer to the hole 36 through which the adjustable fastener 38 is threaded. In this manner, the adjustable fastener may be adjusted to suit the user's preference. The user of the eye shield 10 may form the shield as required to conform to his or her face by pulling on the adjustable fastener 38 until the eye shield 10 fits the contours of the user's face.

In use, the eye shield 10 will tend to bend around the user's face without creasing across the central region 26, which otherwise could impact the user's clear vision.

An advantage of the present invention is that this bending is allowed to occur in more than one plane. This is due in part to the combination of the slits 20, and the slit 46 or cutout 48 to accommodate the user's nose. These slits and/or cutout enable the sections 28, 30, 32, and 34 of regions 22 and 24 to independently slide one over another. The result is a eye shield that serves to deflect thrown or splashed liquids which otherwise could impact the user's eyes. The eye shield 10 also has the advantage of being lightweight and comfortable thus allowing the user to wear the shield comfortably for long periods of time.

It is to be understood that eye shields 10 of the type described may also be 10 adapted to be affixed to or used in conjunction with face masks as depicted in FIG. 3. Additional features (not shown) that could be incorporated into eye shields of the present invention include: spacers or padding added to the shield to absorb and wick perspiration; a darkened band along the top edge, the bottom edge, side edges, as well as any combination to reduce or minimize glare; additional slits for more controlled deformation of the shield; as well as additional fasteners to secure the shield in place.

Although the present invention and its advantages have been described in detail, it should be understood that various changes, substitutions, and alterations can be made therein without departing from the spirit and scope of the invention as defined by the appended claims.

What is claimed is:

1. An adjustable eye shield comprising:
a flexible, transparent material adapted to fit over the eyes and at least partially wrap around the face of a user, the material divided into a plurality of regions, comprising at least a left region, a right region, and a central region;
at least one slit in both the left and the right regions, the slit extending fully across the region thus subdividing the left region into a first and a second section and subdividing the right region into a first and a second section;
at least one hole in each of the first sections of the left and the right regions; and
an adjustable fastener for fastening the eye shield to the head of the user having a plurality of ends, at least one end of the fastener passes through at least one of the holes in the first section of the right region, traverses the slit in the right region, and is fastened to the second section of the right region, at least one other end of the fastener passes through at least one of the holes in the first section of the left region, traverses the slit in the left region, and is secured to the second section of the left region;
wherein the first and second sections of each of the left and right regions are adapted to slide one over the other when the adjustable fastener is pulled.

2. The apparatus of claim 1 wherein the adjustable fastener comprises a loop of material having two ends, wherein one end is secured to one of the second sections and the other end is secured to the other of the second sections.

3. The apparatus of claim 1 wherein the adjustable fastener comprises a first and a second length of material each having a first and a second end, the first length of material is secured at its first end to one of the second sections, the second length of material is secured at its first end to the other second section, and the second ends of each length of material are fastened one to the other.

4. The apparatus of claim 1 wherein the adjustable fastener comprises a cord constructed of a heat-bondable material having a first and a second end, the cord ends being heat-bonded to the second sections of the left and right regions.

5. The apparatus of claim 1 comprising a clear polyester film.

6. The apparatus of claim 1 wherein the adjustable fastener is elastically stretchable.

7. The apparatus of claim 1 wherein the material gradually bends and conforms approximately to the user's face without creasing by manipulation of the adjustable fastener.

8. The apparatus of claim 1 comprising at least one hole in each of the second sections of the left and the right regions through which the ends of the fastener passes prior to being fastened.

9. The apparatus of claim 1 comprising a notch in a lower edge of the central region to allow the eye shield to conform to the face and nose of the user.

10. The apparatus of claim 1 adapted to be attached to a face mask.

11. The apparatus of claim 1 wherein the first sections are located proximate to a top edge of the shield.

12. The apparatus of claim 1 wherein the first sections are located proximate to a bottom edge of the shield.

13. The apparatus of claim 1 wherein the adjustable fastener traverses the slit from one face of the shield to an opposing face of the shield.

14. An adjustable eye shield comprising:
a flattened, flexible, transparent sheet material divided into a plurality of regions, comprising at least a left region, a right region, and a central region;
at least one slit extending fully across the left region subdividing the region into a first and a second section, one of the sections comprising a hole proximate to an edge and distal from the slit;
at least one slit extending fully across the right region subdividing the region into a first and a second section, at least one of the sections comprising a hole;
at least one hole in each of the first sections of the left and the right regions at a location distal from the central region; and
an adjustable fastener having a plurality of ends, a separate end selected from the plurality of ends dedicated to each of the left and right regions so that the dedicated end is affixed to one of the sections within its dedicated region, traverses the slit in the same region, and passes through the hole in the remaining section opposite the slit in the same region;
wherein by manipulation of the adjustable fastener the first and second sections of each of the left and right regions are adapted to slide one over the other causing the material to gradually bend without creasing.

15. The apparatus of claim 14 wherein the adjustable fastener comprises a loop of material having two ends, wherein one end is secured to one of the second sections and the other end is secured to the other of the second sections.

16. The apparatus of claim 14 wherein the adjustable fastener comprises a first and a second length of material each having a first and a second end, the first length of material is secured at its first end to one of the sections of the regions, the second length of material is secured at its first end to one of the sections of the other region, and the second ends of each length of material are fastened one to the other.

17. The apparatus of claim 14 wherein the adjustable fastener comprises a cord constructed of a heat-bondable material having a first and a second end, each of the cord ends being heat-bonded to one of the sections in each of the left and right regions.

18. The apparatus of claim 14 comprising a clear polyester film.

19. The apparatus of claim 14 wherein the adjustable fastener is elastically stretchable.

20. The apparatus of claim 14 comprising a notch in a lower edge of the central region to allow the eye shield to conform to the face and nose of the user.

21. The apparatus of claim 14 adapted to be attached to a face mask.

22. The apparatus of claim 14 wherein the first sections are located proximate to a top edge of the shield.

23. The apparatus of claim 14 wherein the first sections are located proximate to a bottom edge of the shield.

24. The apparatus of claim 14 wherein the adjustable fastener traverses the slit from one face of the shield to an opposing face of the shield.
